(19) United States
(12) Patent Application Publication
(10) Pub. No.: US 2015/0217104 A1
(43) Pub. Date: Aug. 6, 2015

(54) CONNECTOR FOR PRODUCING A FLUID CONNECTION TO A SECOND CONNECTOR, CONNECTOR SYSTEM, AND METHOD FOR PRODUCING A FLUID CONNECTION

(71) Applicant: Fresenius Kabi Deutschland GmbH, Bad Homburg (DE)

(72) Inventors: Martin Biehl, St. Wendel (DE); Ilka Sternheimer, Frankfurt (DE)

(73) Assignee: Fresenius Kabi Deutschland Gmbh, Bad Homburg (DE)

(21) Appl. No.: 14/424,537

(22) PCT Filed: Aug. 16, 2013
(86) PCT No.: PCT/EP2013/067119

§ 371 Date: Feb. 27, 2015

Related U.S. Application Data
(60) Provisional application No. 61/695,523, filed on Aug. 31, 2012.

(30) Foreign Application Priority Data
Aug. 28, 2012 (EP) 12181957.7

(51) Int. Cl. A61M 39/10

(52) U.S. Cl. CPC A61M 39/10; A61M 2039/1061

(57) ABSTRACT

A connector for producing a fluid connection to a second connector, wherein the connector can, in at least one connector area, form a weakened structure, in order to be able to break open the connector in the connector area and in this way produce a fluid connection to the second connector. A partial area of the connector area is designed in such a way that the weakened structure in the partial area can be generated by covering or spraying the connector area with a medium.

The invention relates to a connector according to the preamble of claim 1 for producing a fluid connection to a second connector, and to a connector system according to the preamble of claim 11, having a first connector and a second connector via which a fluid connection can be produced between at least two fluid-conveying systems, and furthermore to a method according to the preamble of claim 17 for producing a connection between at least two fluid-conveying systems by means of a first connector and a second connector.

In its intended use, a connector of the type mentioned above, which serves to produce a fluid connection to a second connector, closes off a fluid-conveying and, for example, sterile system and, in at least one connector area, makes available a weakened structure in the form of a partial area of said connector area, so as to be able to break open or break through the connector in this connector area (with destruction of the weakened structure) and in this way be able to produce the fluid connection to the second connector.

The connector can be suitable, as a medical connector, in particular for the sterile connection of hose ends, syringes, needles and other medical components. In this case, one of the two connectors is usually configured as a so-called male connector, to which a female connector is assigned as further connector, with the male connector being inserted into the female connector. The respective connector can also be provided with a protective cap.

Specifically, the connector can close off a sterile system by being assigned to the sterile system as a connection element that does not impair the sterility of this system. By producing a fluid connection to a second connector, which is assigned as connection element to a further fluid-conveying system and closes off this system, a fluid connection between said two systems is permitted. In this case, provision is generally made that the second connector also closes off a sterile system. The resulting fluid connection is then a sterile connection between a first connector and a second connector, which each close off a sterile system.

However, the respective connector can also be assigned to a non-sterile system in order to close the latter. It is also possible that only the first connector closes off a sterile system, while such a requirement does not apply to the second connector to be connected thereto.

By means of a fluid connection of this kind, it is possible, for example, for biological and medical substances to be transferred from one closed (sterile) system to another one. This applies, for example, to the transfer of blood and/or blood constituents into a bag for storage, the transfer of storage solutions for blood and blood products, and the transfer of solutions into other types of systems.

DE 199 60 226 C1 discloses a connection system for connecting at least two systems, said connection system comprising a male connector part, which forms a closed end of a first sterile, fluid-conveying system, and a female connector part, which forms a closed end of a second sterile, fluid-conveying system, and being designed to connect the two sterile systems to each other in an aseptic or sterile manner, such that fluid exchange can take place between the two systems. For this purpose, the two connector parts each have a predetermined break point, the latter lying one over the other when the connector parts are joined together as intended, such that said predetermined break points form a common predetermined break point in the interior of the resulting fluid-conveying system and can be broken off together in order to produce the fluid connection between the two closed systems. To ensure the sterility, particularly during the production of the fluid connection by connecting the two connector parts, a disinfectant is applied to the mutually touching contact faces of the connector parts. This disinfectant can additionally have adhesive properties, e.g. through use of a disinfecting adhesive, such that the disinfectant also serves for the (permanent) connection of the two connector parts.

The problem addressed by the invention is that of further improving a connector of the type mentioned at the outset, a corresponding connector system, and a method for producing a fluid connection between at least two sterile systems.

As regards the connector, this problem is solved by the features of claim 1.

According to the latter, the connector area to be weakened in a partial area is designed in such a way that its weakened structure can be generated by at least partially covering or irradiating the connector area with a medium.

That is to say, in the connector according to the invention, a weakened structure in the connector area is initially formed only by a partial area of said connector area, designed specifically for interaction with a medium, being weakened when the medium acts on the connector area. In particular, a mechanically weakened structure can be generated here. Outside said partial area, the connector area is advantageously inert to the action of the medium and thus maintains its original stability.

The medium can, for example, be a fluid or pasty medium with which the connector area is covered, in order to generate the weakened structure there in a partial area. Moreover, the medium can be a radiation, in particular electromagnetic radiation, with which the connector area is irradiated in order to weaken it in a partial area.

Since the weakened structure of the connector can be generated by covering or irradiating a connector area with a medium, which advantageously takes place directly before or during the connection to an associated second connector, it is easily possible to achieve sufficient stability of the connector, in particular also of the connector area provided with the weakened structure, during storage and transport.

Provision can be made in particular that the connector area to be weakened in a partial area has in some areas a material that forms the weakened structure only in the one partial area when the connector area is covered or irradiated as a whole with a medium. For this purpose, the connector area can be made from a first material and from a second material different than the latter, the second material being less resistant to the medium than is the first material, such that, upon contact with said medium, the connector area is weakened in the partial area made from the second material, e.g. by a chemical reaction being triggered in said second material by the medium or specifically by stress cracks being induced.

Thus, the connector area can be made from two different plastics, of which the second one is chosen in such a way that, by contact with the medium, it generates a weakened structure inside the connector area, which otherwise consists of the first material. Such a connector area can be produced by the so-called two component technique by molding, in particular injection molding, in one operation in one mold.

Examples of suitable materials which form a weakened structure when covered or irradiated with a suitable medium, e.g. a medium triggering a chemical reaction or forming stress cracks, are polymethyl methacrylate, polycarbonate, polyethylene, polystyrene and polysulfone.

As the medium for covering or irradiating the connector area in order to generate a weakened structure there, it is possible in particular to use a disinfectant, e.g. in the form of a fluid or pasty disinfectant or in the form of a disinfecting radiation, such as microwave or ultraviolet (UV) radiation. This allows a weakened structure to be generated in the connector area without an additional operating step, since the covering or wetting or irradiating of the connector area with a disinfectant is in any case generally provided (directly) before or during the production of a connection to a second connector, particularly in the case of a sterile connection. The disinfectant here assumes a dual function, namely on the one hand, the disinfection of the connector during the connection to a second connector and, on the other hand, the formation of a weakened structure on the connector area. Moreover, apart from a disinfection of the connector, as is necessary in any case, no additional energy input into the connector or connector area is needed.

Generally, variants of the invention are advantageous in which the weakening of the partial area of the connector area takes place by the action of the medium and without additional energy input.

The medium can take the form, for example, of all the disinfectants that are officially approved (e.g. by the Paul-Ehrlich Institute for Germany or Europe). In particular, the medium can be chosen, for example, from a group comprising the (low molecular weight) alcohols, polyols, fat-containing emulsions and buffer solutions, the latter in particular with a pH value of 1 to 3 or of 11 to 13.

The covering or wetting with a disinfectant can take place, for example, by spraying the connector with the disinfectant or by immersing it in a bath of disinfectant.

The effect of the medium on the partial area of the connector area can specifically be such that a chemical reaction is triggered in the partial area of the connector area that is to be (mechanically) weakened, and, for example in the case of the connector area being irradiated with microwave radiation or UV radiation, the medium itself does not necessarily have to be a reaction partner. However, provision can also be made that the medium, e.g. in the form of a fluid or pasty medium, reacts (chemically) with the material in the partial area of the connector area and, in this way, the weakened structure is generated. A chemical partial dissolution of the material in the partial area of the connector area, e.g. plastic, by the medium, particularly in the form of a disinfectant, can also take place.

Furthermore, the medium can trigger a chemical reaction which does not itself lead directly to a weakening of the connector area, but which releases heat, the effect of the latter weakening the connector area in the partial area intended for this purpose.

In the present case, the weakening of the connector area takes place only in the partial area of the connector area designed for this purpose, e.g. in terms of its material. The other portions of the connector area are by contrast not substantially affected by the action of the medium. That is to say, outside said partial area, the connector area is advantageously inert to the action of the medium, e.g. on account of the material there.

Before the partially weakened connector area is broken through in order to produce a fluid connection to the second connector, the connector is impermeable to fluid. This means in particular that the connector area is initially impermeable to fluid before being covered with the medium that forms the weakened structure, so as to permit the formation of a (sterile) closed system, and it advantageously remains impermeable to fluid after being covered with the medium and after the associated formation of a weakened structure, until the connector is broken open at the weakened structure.

The connector area to be weakened in a partial area can therefore be in particular a closure area of the connector that closes the connector in a fluid-tight manner, e.g. by closing a fluid-conveying channel of the connector. After the connector is broken open in said closure area at the weakened structure, said channel can then serve to produce the fluid connection to a second connector.

Here, the closure area can be made from at least two materials, of which one is weakened when covered or irradiated with a medium provided for this purpose, such that the closure area forms a weakened structure in a corresponding partial area. By contrast, the other portions of the connector area that are made from another material are not weakened.

The connection of the connector to an associated second connector can be effected by plugging them together, in particular by plugging them together axially, and/or by twisting them relative to each other, as a result of which it is possible to produce, for example, a form-fit connection, e.g. a locking connection, a bayonet connection, etc., or else a screw connection, e.g. using a suitable thread. The connectors to be connected to each other can also be screwed together using an additional connecting part, e.g. in the form of a union nut.

The breaking open or through of the connector area at its weakened structure can take place on the one hand directly during the connection of the connector to a second connector, e.g. by piercing the weakened structure, by radial stressing of the weakened structure during or after a constriction, or by torsional stresses when twisting the two connectors relative to each other. On the other hand, however, the connector area can also break along the weakened structure only after connection to a second connector, e.g. manually or by means of a tool.

A projection for piercing the weakened structure of one connector, and provided for this purpose on the second connector to be connected, can itself in turn have a weakened structure, so as to be able to break off this projection and in this way also, if appropriate, allow fluid to pass through the second connector.

There are many possible contours of the weakened structure that permit simple breaking-through of the connector area in order to produce a fluid connection to a second connector. Advantageously, the weakened structure is designed, for example, in such a way that, when the connector area is broken through, it allows portions of the connector area that do not belong to the weakened structure, e.g. portions resistant to disinfectant, to press against an outer wall of the resulting connector system.

A connector system, for producing a fluid connection between at least two fluid-conveying and in particular sterile systems via two connectors, of which each is assigned to one of the two systems, is characterized by the features of claim 11. Advantageous embodiments of the connector system are set forth in the claims dependent on said claim.

A method for producing a connection between at least two fluid-conveying and in particular sterile systems by means of a first connector and a second connector, which each close off a sterile system, is set forth in claim 17. Advantageous developments of the method are set forth in the claims dependent on said claim.

According to a specific embodiment of the method, provision can in particular be made that the one (male) connector can be inserted into the other (female) connector in order to produce a connection in this way. The latter can be a form-fit connection, for example, in particular in the form of a locking connection, e.g. in which the one connector engages behind an undercut on the other connector, or in which the two connectors are twisted relative to each other to produce a bayonet or screw connection. The connection can in particular be configured such that the two connectors cannot be released again from each other by a (rectilinear) longitudinal or axial force.

When producing the connection between the two connectors, a respective connector area can also be broken open to allow fluid to pass through between the two connectors. Alternatively, a respective connector area can be broken open, e.g. manually or by means of a tool, only after the connection has been produced.

The invention is described in detail below on the basis of illustrative embodiments and with reference to the drawings, in which:

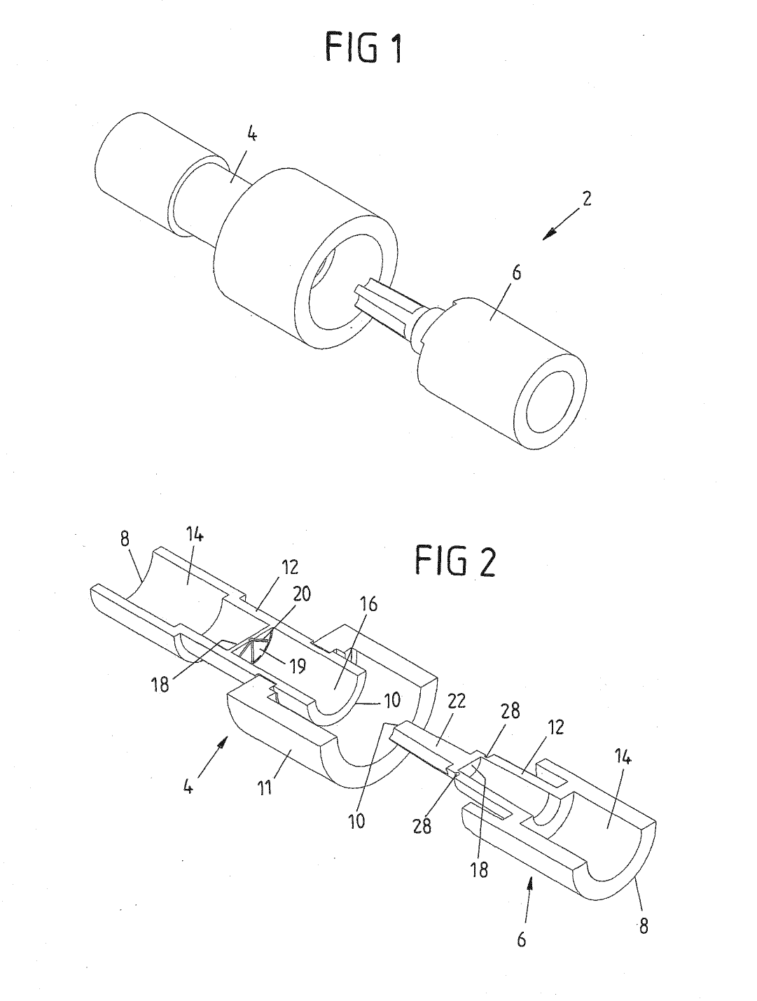

FIG. 1 shows a perspective view of a connector system with a first connector and a second connector according to a first embodiment of the invention in the unconnected state;

FIG. 2 shows a cross section through the connector system from FIG. 1;

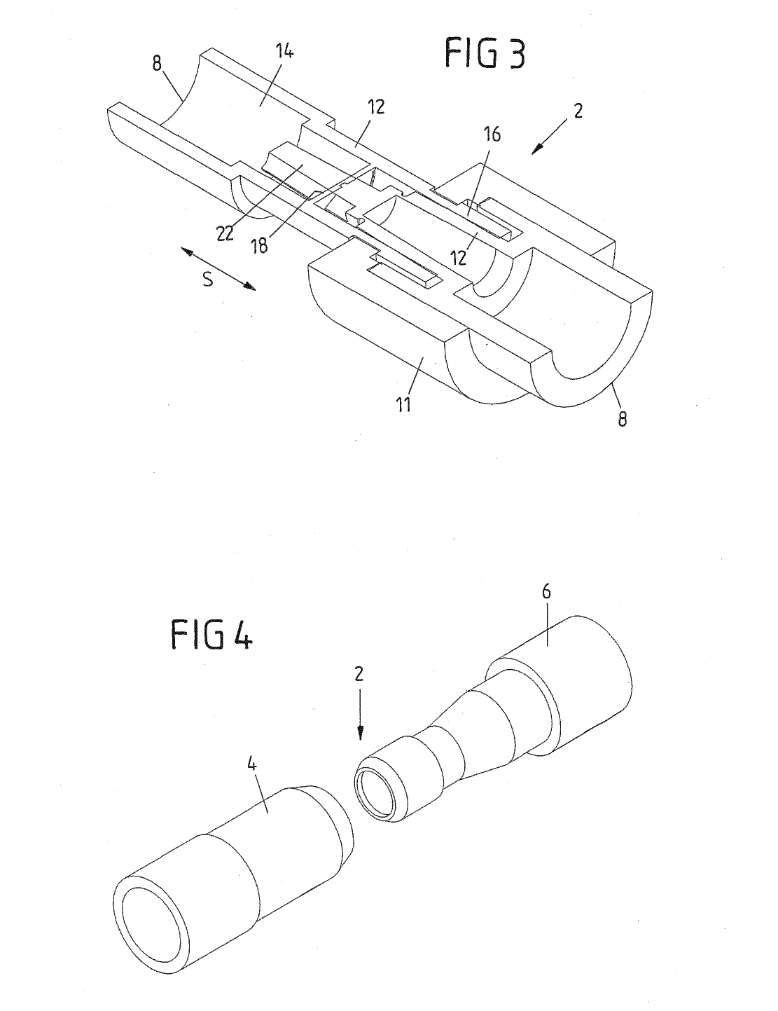

FIG. 3 shows a cross section through the connector system from FIG. 1 in the connected state;

FIG. 4 shows a perspective view of a connector system with a first connector and a second connector according to a second embodiment of the invention in the unconnected state;

FIG. 5 shows a cross section through the connector system from FIG. 4;

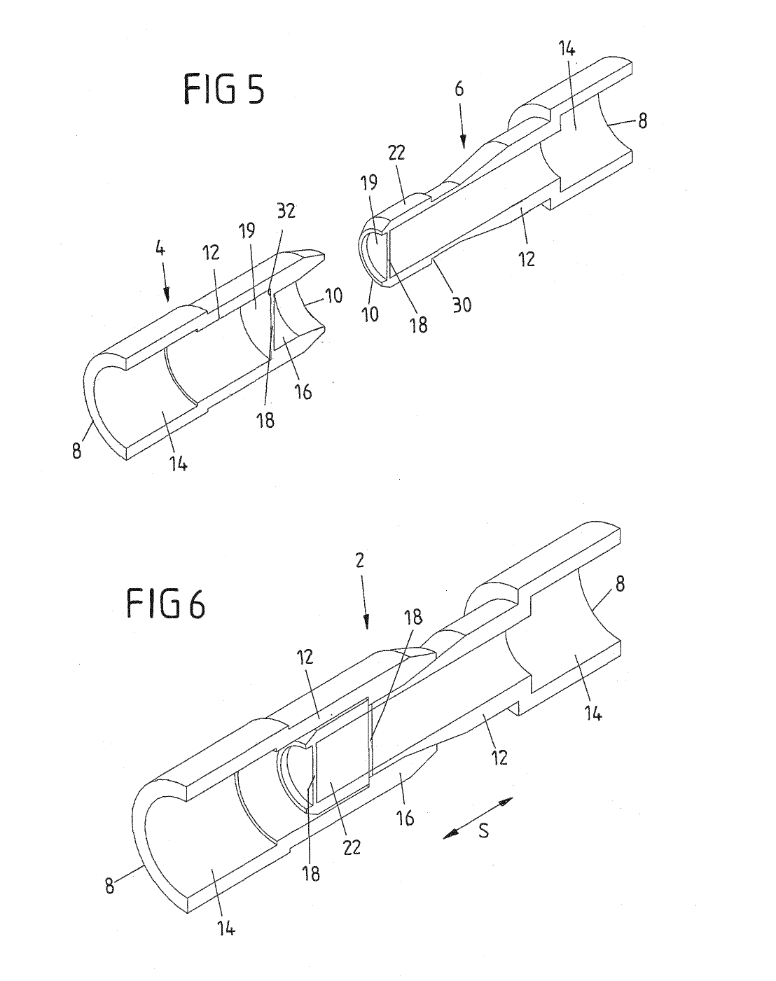

FIG. 6 shows a cross section through the connector system from FIG. 4 in the connected state;

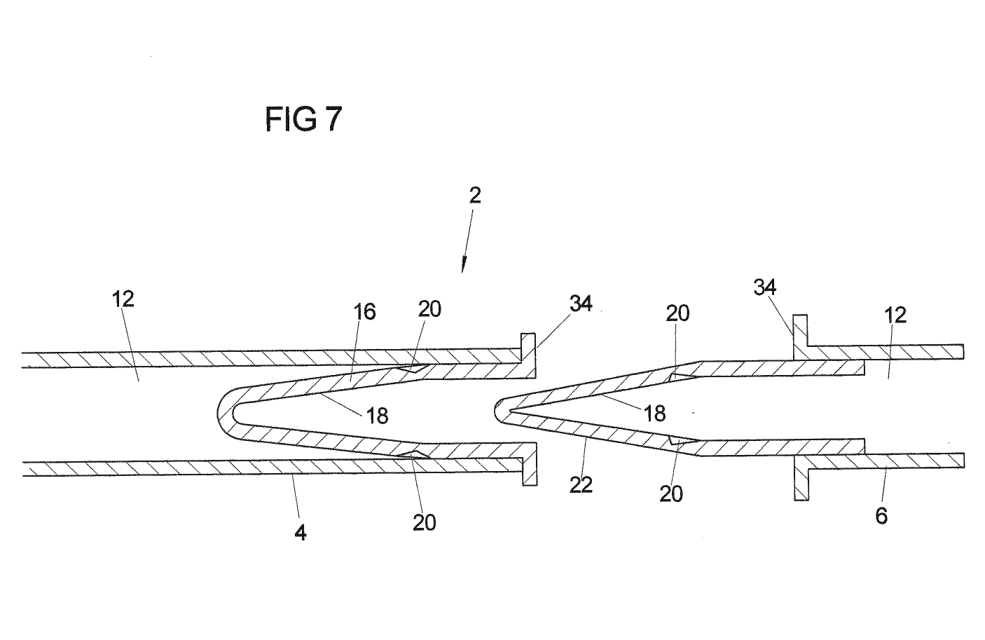

FIG. 7 shows a schematic cross-sectional view of a connector system with a first connector and a second connector according to a third embodiment of the invention in the unconnected state;

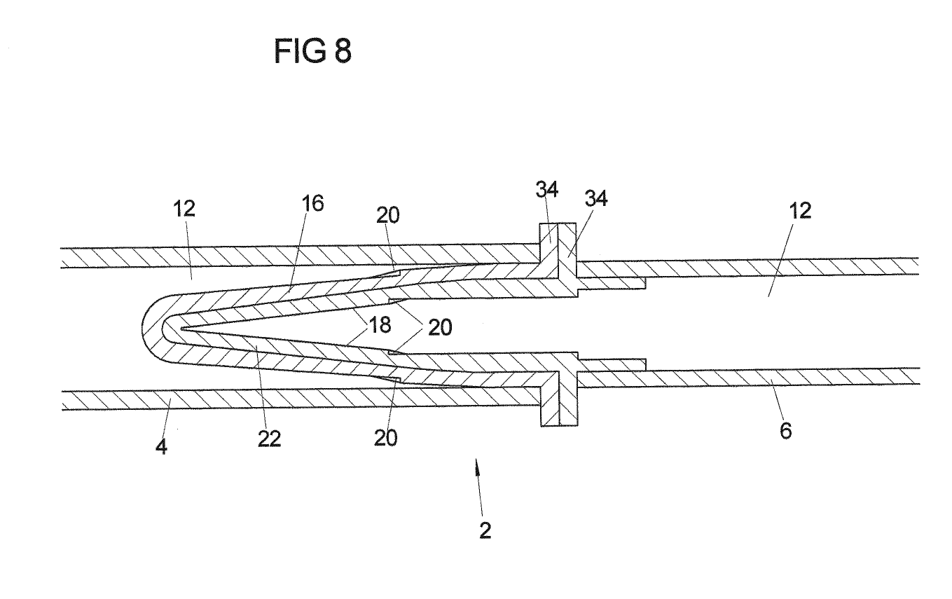

FIG. 8 shows the connector system from FIG. 7 in the connected state;

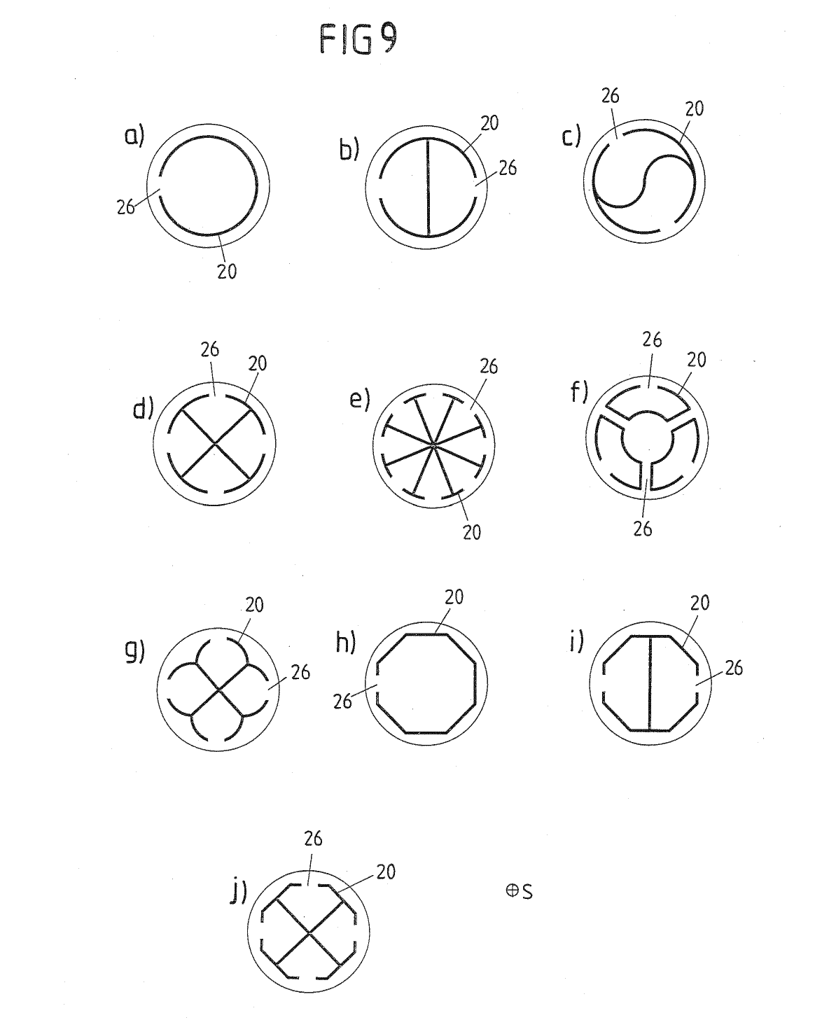

FIG. 9 shows various embodiments of a weakened structure as a partial area of a connector area of a connector; and

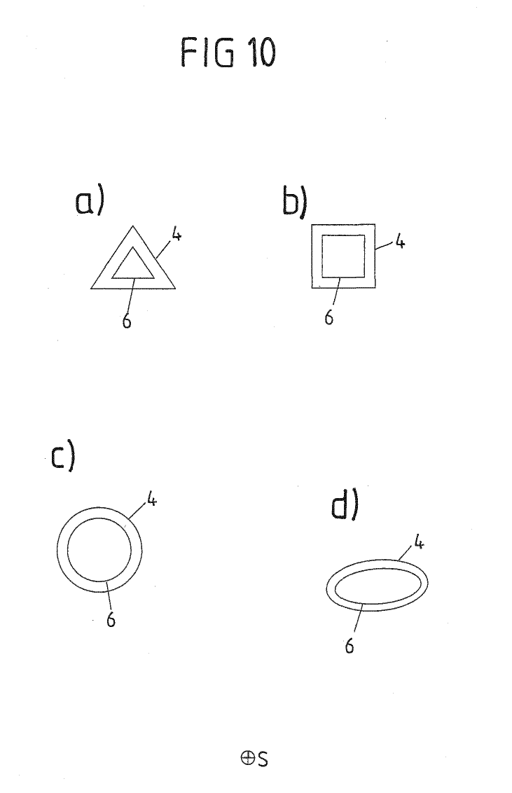

FIG. 10 shows various embodiments of the cross-sectional geometry of a connector.

A connector system 2 in a first embodiment is shown in FIGS. 1 to 3. The connector system 2 serves to produce a connection between two fluid-conveying and optionally sterile systems (not shown in the figures). In particular, the connector system 2 allows two sterile systems to be connected to each other in a sterile manner even in a non-sterile environment. Thus, the connector system 2 can be used for medical applications, for example for the sterile connection of syringes, needles, hoses, medical appliances and other fluid-conveying medical devices.

The connector system 2 comprises a first connector 4 and a second connector 6, which can be connected to each other (axially) along a flow direction S.

Before the connector system is described in detail below with reference to FIGS. 1 to 3, and in modified forms with reference to FIGS. 4 to 8, we will first provide a general explanation of the basic structure of the first connector 4 according to the present invention. The first connector 4 serves for attachment to an associated fluid-conveying and optionally sterile system and, for this purpose, has a channel 12 that is to be brought into fluid connection with the associated fluid-conveying system (see FIGS. 1 and 2).

Moreover, the first connector 4 is designed to produce a fluid connection to the second connector 6, in particular to produce in this way a fluid connection between the fluid-conveying system assigned to the first connector 4 and a fluid-conveying system assigned to the second connector 6. The fluid-conveying connection to the second connector 6 also in particular entails the production of a fluid connection between the channel 12 of the first connector 4 and the second connector 6. Thus, in the illustrative embodiment, the channel 12 can produce the fluid-conveying connection between the fluid-conveying system assigned to the first connector 4 and the further fluid-conveying system assigned to the second connector 6.

According to FIG. 2, the channel 12, and therefore the first connector 4, is closed by a connector area 18 in the form of a closure area, which is formed here by a disk 19, for example.

The connector area 18 closing the first connector 4, specifically the channel 12 thereof, has a partial area 20 that can be weakened by the action of a suitable medium. This partial area 20 can be formed, for example, by virtue of the connector area 18 being made, in the partial area 20, from another material than outside said partial area 20. If a suitable medium, e.g. a fluid or pasty medium, or a medium formed by radiation, particularly in the form of a disinfectant, now acts on the connector area 18, the latter is thereby weakened in a deliberate manner in the partial area 20 designed accordingly for this purpose. The resulting weakened structure can then be broken open, particularly upon connection of the two connectors 4, 6, so as to produce the desired fluid connection. Before the connector area 18 is broken open, the first connector 4 closes off the associated fluid-conveying system in a fluid-tight manner.

The design of the connector system 2 shown in FIGS. 1 to 3 is now explained in detail:

The first connector 4 and the second connector 6 each have a first connection end 8 and a second connection end 10. The first connection end 8 serves for the attachment of the first connector 4 and of the second connector 6 in each case to a fluid-conveying and optionally sterile system. The first connector 4 and the second connector 6 are initially impermeable to fluid and close off the respective sterile system. The respective second connection end 10 of the first connector 4 and of the second connector 6 is provided for producing a connection between the two connectors 4, 6. This connection is configured here, for example, as a releasable connection. For this purpose, a union nut 11 with an inner thread is provided on the second connection end 10 of one connector 4, 6, and a corresponding outer thread (not shown) is provided on the second connection end 10 of the other connector 6, 4. The inner thread and the outer thread can be replaced or supplemented by a locking arrangement, as a result of which an at least axially non-releasable connection can also be produced. The locking can take place using a tool.

The first connector 4 has a channel 12 which extends along the flow direction S and via which the fluid connection can be produced both to the associated fluid-conveying system and also to the second connector 6. The channel 12 opens at one end into a rear connector element 14, which forms the first connection end 8, and opens at the other end into a front connector element 16, in the present case a female connector element, which forms the second connection end 10. Thus, as regards its function in the connector system 2, the first connector 4 is a female connector in the illustrative embodiment. The rear connector element 14 can be male or female for the attachment of a fluid-conveying system.

The channel 16 is closed by a connector area 18 acting as closure area, such that the first connector 18 closes off the associated fluid-conveying system. That is to say, the rear connector element 14 and the front connector element 16 are separated from each other (impermeable to fluid) by the connector area 18 that is arranged in the channel 12 and that acts as closure area. In the illustrative embodiment, the connector area 18 is designed as a disk 19, which in the present case extends substantially transversely with respect to the flow direction S across the cross section of the channel 12. Moreover, in the illustrative embodiment, the connector area 18 is shaped or formed in one piece with further portions of the connector, e.g. portions forming the channel 12 and/or the connection ends 8, 10.

The connector area 18 is in this case designed to form a weakened structure in the first connector 4, in a partial area 20 of the connector area 18. The weakened structure can be generated by covering the connector area 18 (partially or completely) with a medium. The resulting weakened structure 20 is designed such that it can be broken through by the action of an external force and, in this way, the channel 12 can be opened to produce a fluid connection. In particular, this creates, inside the connector, a fluid connection between the rear connector element 14 and the front connector element 16.

For the stated purpose, the connector area 18 has, as closure area, at least one first material and at least one second material that have a different resistance with respect to the medium that is to be applied. The second material (lower resistance) forms the partial area 20 of the connector area 18 to be weakened and is distributed as a pattern in the first material (higher resistance). The pattern of the second material defines the geometry of the weakened structure that is to be formed. The remaining portions of the connector area 18 are made from the first material.

The first material can be, for example, polyamide (PA), polyether ether ketone (PEEK), polyethylene terephthalate (PET), polypropylene (PP) or polyphenylene sulfide (PPS). The first material can also correspond to the material of at least some parts of the rest of the first connector 4 (outside the closure area). The second material can be, for example, polymethyl methacrylate (PMMA), polycarbonate (PC), polyethylene (PE), polystyrene (PS) or polysulfone (PSU). The second material is distinguished by the fact that it is less resistant to the medium than is the first material.

The medium can be a disinfectant for example, including in particular all the disinfectants officially approved by the Paul-Ehrlich Institute, e.g. Diosol® or Descoderm®, a polyol, a fat-containing emulsion, a (low molecular weight) alcohol or a buffer solution, in particular with a pH value of 1.5 to 2.5 or of 11.5 to 12.5. Moreover, the medium can be an advantageously disinfecting radiation, e.g. microwave radiation or UV radiation.

The lesser resistance of the second material has the consequence that, when the second material comes into contact with one of the aforementioned media, the second material is partially dissolved and/or otherwise weakened by the medium. The medium can trigger a chemical reaction in the second material and can optionally react with the second material. The weakened second material then forms a weakened structure in the partial area 20 of the connector area 18. The weakened structure may, for example, tend to form stress cracks if the connector area 18 is exposed to mechanical stress. Thus, under the action of an external force, the connector area 18 is able to free a fluid connection through the channel 12. By contrast, without the action of an external force, the connector area 18 with the weakened structure remains advantageously impermeable to fluid.

By comparison, the stability of the first material of the connector area 18 is not appreciably affected by contact with the medium.

The partial area 20 forming the weakened structure can adopt different geometries. Some examples are shown in FIGS. 9a to 9j, which are described further below. Alternatively, other suitable geometries are also conceivable. Advantageously, the partial area 20 forming the weakened structure has elements that extend in the circumferential direction and/or radially with respect to the flow direction S. The geometry of the partial area 20 is preferably chosen such that the resulting weakened structure does not divide the connector area 18 into two or more portions 24 completely separate from each other. Instead, the partial area 20, or the weakened structure resulting therefrom, preferably has interruptions 26 which ensure that, when there is permeability of fluid through the channel 12 after piercing of the connector area 18, i.e., in the illustrative embodiment, when a fluid connection is present between the rear connector element 14 and the front connector element 16, preferably all of the portions 24 of the connector area 18 remain (permanently) connected to the channel 12 and are preferably pressed against the inner boundary wall of the latter. This prevents individual portions 24 of the connector area 18 getting, as freely movable elements, into the fluid connection. As a result, there should be no sharp edges in the channel 12, nor should particles come loose from the connector 4 and get into the channel 12.

The opening generated in the channel 12, at the site of the broken-through connector area 18, should be large enough that it does not impede the through-flow of the fluid conveyed therein. In the case of blood transport, for example, it should therefore allow blood cells to pass through.

The second connector 6 likewise has, like the first connector 4, a channel 12 which extends along the flow direction S and which serves to produce a fluid connection to the associated fluid-conveying system or to the other first connector 4 and which, in the illustrative embodiment, opens at one end into a rear connector element 14, which forms the first connection end 8, and into a front connector element 22, which forms the second connection end 10. The second connector 6 differs from the first connector 4 mainly in that the channel 12 ends with a front connector element 22 in the form of a male connector element. As regards its function in the connector system 2, the second connector 6 is thus a male connector. The rear connector element 14, which serves for attachment to a fluid-conveying and in particular sterile system, can be male or female.

Analogously to the first connector 4, the rear connector element 14 and the front connector element 22 of the second connector 6 are separated from each other by a connector area 18 arranged in the channel 12. The connector area 18 is arranged here at the border between the front connector element 22 and the channel 12.

Accordingly, the connector area 18 of the second connector 6 again forms a closure area for closing the channel 12, such that the connector 6 can close off the associated fluid-conveying system in a fluid-tight and optionally sterile manner.

The connector area 18 of the second connector 6 is therefore intended, on the one hand, to separate the rear connector element 14 and the male connector element 22 from each other in a manner impermeable to fluid, and, on the other hand, to deliberately, i.e. under the action of an external force, free a fluid connection via the channel 12, i.e., in the illustrative embodiment, between the rear connector element 14 and the male connector element 22.

In the present case, the connector area 18 of the second connector 6 has, as closure area, a disk 19 which, in the illustrative embodiment, extends substantially transversely with respect to the flow direction S across the cross section of the channel 12.

In the present case, the connector area 18 in the form of a closure area also comprises a predetermined break point 28, in the illustrative embodiment a ready-made break point 28. The predetermined break point 28 is here displaced with respect to the disk 19 in the flow direction S, specifically in such a way that the disk 19 lies closer to the front connector element 22 than to the predetermined break point 28. The predetermined break point 28 is the result of a material taper in the outer surface of the second connector 6, which material taper extends peripherally and transversely with respect to the flow direction S. Alternatively or in addition, the predetermined break point 28 can also be provided, for example, on the inner surface of the second connector 6.

The predetermined break point 28 is configured in such a way that, when the first connector 4 and the second connector 6 are connected, it does not lead to the second connector breaking (FIG. 3), but it can be broken under the action of a force transverse to the flow direction S (manually or by means of a tool), in order to form a fluid connection between the first connector 4 and the second connector 6.

Alternatively, the connector area 18 of the second connector 6 can be designed such that the connector area 18 forms a weakened structure only upon contact with a suitable medium.

In the embodiment shown in FIGS. 1 to 3, the first connector 4 is a female connector and the second connector 6 is a male connector. However, in one variant, the first connector 4 can also be male and the second connector 6 female.

A connector system 2 in a second embodiment is shown in FIGS. 4 to 6. The second embodiment differs from the first embodiment particularly in that the front (male) connector element 22 of the second connector 6 has an undercut 30, and the front (female) connector element 16 of the first connector 4 has an associated abutment surface 32. The abutment surface 32 is formed by the channel 12 of the first connector 4 having a greater internal diameter than the adjoining front connector element 16. According to one embodiment, the internal diameter of the channel 12 of the first connector 4 corresponds at least to the external diameter of the front connector element 22 of the second connector. This has the effect that, when the front male connector element 22 of the second connector 6 is inserted into the front female connector element 16 of the first connector, a radial stress is built up on the front connector element 22 or on the connector area 18 of the second connector 6. In the connected state (FIG. 6), the front connector element 22 of the second connector 6 is again free of stress or subject to a lesser radial stress. Through such changes in stress, a weakened structure present in the connector area 18, and formed optionally by contact with a medium, can be broken open.

The connection between the first connector 4 and the second connector 6 of the connector system 2 according to the second embodiment is not releasable by a force acting in the axial direction. This is a result of the above-described geometry, according to which the undercut 30 engages behind the abutment surface 32.

According to one variant, a locking arrangement is provided on the inner face of the second connection end 10 of the first connector 4 and on the outer face of the second connection end 10 of the second connector 6, for non-releasable connection of the first connector 4 and of the second connector 6.

In the present case, both the first connector 4 and also the second connector 6 can have a connector area 18 which forms a weakened structure in a partial area only upon contact with a suitable medium. In the present case, the connector area 18 of the first connector 4 extends, for example in the form of a disk, at the boundary between the channel 12 and the front connector element 16, substantially transversely with respect to the flow direction S. The connector area 18 of the second connector 6 closes off the second connection end 10 of the front connector element 22.

According to a modified configuration, the connector area 18 of the first connector 4 or of the second connector 6 can have a ready-made predetermined break point instead of a partial area 20 that is to be weakened.

The first and second embodiments of the connector system 2 are not limited to the respectively described type of connection of the first connector 4 and the second connector 6. Instead, for both embodiments, it is possible for the connectors 4, 6 to be connected not just releasably, e.g. by screwing, but also non-releasably, for example by form-fit engagement.

FIGS. 7 and 8 show a detail of a connector system 2 in a third embodiment. The third embodiment differs from the first and second embodiments firstly in that the connector areas 18 of the first connector 4 and second connector 6, to be brought into contact with a medium in order to form a weakened structure, do not comprise a disk-shaped closure area. In the present case, the connector areas 18 are instead formed by (closed) end portions of the front connector elements 16, 22 of the first connector 4 and of the second connector 6, and they have a substantially conical shape. The shape and dimensions of the connector elements 16, 22 of the first and second connectors 4, 6 to be connected to each other are here adapted to each other in such a way that, in the connected state of the connector system 2 (FIG. 8), the outer surface of the front male connector element 22 of the second connector bears on the inner surface of the front female connector element 16 of the first connector 4.

Prior to the formation of the weakened structure in the respective connector area, the connectors 4, 6 are impermeable to fluid, so as to allow a fluid-conveying and optionally sterile system to be closed off in a fluid-tight manner. Advantageously, the connectors 4, 6 remain impermeable to fluid even after coverage with the medium and after the associated formation of a weakened structure, until the connectors 4, 6 are broken open at the weakened structures.

In the present case, the partial area 20 belonging to a respective connector area 18, and to be weakened by the medium, mainly extends peripherally on a portion of the front female connector element 16 and of the front male connector element 22 spatially delimited along the flow direction S. Preferably, the respective partial area 20 does not extend about the entire circumference of the respective connector element 16, 22. This is intended to prevent a situation where, when the weakened structure is broken open, part of the respective connector element 16, 22 could separate completely from the rest of the connector 4, 6 and could obstruct the fluid connection thereby produced.

In the connected state, the connector areas 18 of the first connector 4 and of the second connector 6 are preferably located at the same level in the flow direction S, such that they together define a common connector area. Thus, the common connector area can be broken open after formation of a weakened structure in a partial area 20 of the connector areas 18, for example by bending the connector system 2.

Advantageously, the first connector 4 and the second connector 6 each have an abutment 34 which shows a user whether the connector elements 16, 22 are bearing on each other as intended. In the connected state, the abutments 34 of the first connector 4 and of the second connector 6 rest on each other.

The connectors 4, 6 of the connector system 2 shown in FIGS. 7 and 8 are connected to each other by a force fit. Alternatively or in addition, connection elements can be provided on the connectors 4, 6 in order to permit a releasable or non-releasable form-fit connection or other kind of connection. According to a variant not shown here, one of the two connectors 4, 6 can also have a ready-made predetermined break point 28 instead of a partial area 20 forming the weakened structure.

In the present case, the connector elements 14, 16, 22 of the connectors 4, 6 have a cone shape. For example, these connector elements 14, 16, 22 are Luer connectors according to ISO 594. For uses outside medicine, e.g. in biotechnology, connectors with larger dimensions can also be provided.

The possible geometries of the partial area 20 to be weakened, and of the resulting weakened structure, are described in detail below with reference to FIGS. 9a to 9j.

According to one variant, the partial area 20 to be weakened forms a ring, in particular of circular shape, with at least one interruption 26 (FIG. 9a). In addition, a radially extending line can be provided segmenting the ring, e.g. halving it, in which case a further interruption 26 can also be provided. The line can be straight (FIG. 9b) or curved, e.g. S-shaped (FIG. 9c). Alternatively, the ring is divided into parts, in particular identical parts, by a number of radially extending lines, e.g. two (FIG. 9d) or four (FIG. 9e), which lines are advantageously at least partially straight, such that the ring is accordingly interrupted four times (FIG. 9d) or eight times (FIG. 9e). According to a further variant, the partial area 20 to be weakened forms a ring interrupted in each case three times in the radial direction and the circumferential direction (FIG. 9f). In another embodiment, the partial area 20 is provided by at least two radially extending lines arranged perpendicular to each other, wherein two adjacent ends of the radial lines are in each case connected by arc-shaped lines that extend in the circumferential direction and that each have an interruption 26 (FIG. 9g). According to a further variant, the partial area 20 to be weakened forms a regular polygon, e.g. an octagon, with an interruption 26 (FIG. 9h). It is also conceivable to have one (FIG. 9i) or two (FIG. 9j) radially extending lines dividing the polygon into (equal) parts, in which case two (FIG. 9i) or four (FIG. 9j) interruptions 26 can accordingly be provided.

FIGS. 10a to 10d show various embodiments for the cross-sectional geometry of the connectors 4, 6. In addition to a triangular (FIG. 10a) and a square (FIG. 10b) cross-sectional geometry, further polygonal shapes are possible, e.g. generally in the form of a polygonal cross-sectional geometry. It is also conceivable for the cross section to be arc-shaped, e.g. round (FIG. 10c) or elliptic (FIG. 10d). The connectors 4, 6 preferably have a round cross-sectional geometry.

The geometry of the weakened structure and the cross-sectional geometry of the connector 4, 6 are chosen, for example, depending on the size of the connector 4, 6 and the field of use of the connector 4, 6.

A method for producing a connection between the first connector 4 and the second connector 6 of the connector system 2 is explained below using the example of the first embodiment shown in FIGS. 1 to 3.

In a first step, the first connector 4 and the second connector 6 are attached to a fluid-conveying and optionally sterile system, in the illustrative embodiment via the respective first connection end 8. The first connector 4 and the second connector 6 are initially impermeable to fluid and thus close off the respective fluid-conveying system. The first connector 4 and the second connector 6 are initially present in a state in which they are not connected to each other (see FIGS. 1 and 2).

In a second step, the first connector 4 and optionally the second connector 6, and in particular also the connector area 18 of the first connector 4, are covered or irradiated with a medium, advantageously a medium having a disinfecting action. The covering can be carried out by spraying. Alternatively, the respective connector 4, 6 can also be immersed in the medium. This step has the purpose of bringing the second material of the connector area 18 of the first connector 4 into contact with the medium, in order to generate a weakened structure there. At the same time, the first connector 4 and the second connector 6 can be disinfected at the surfaces. Examples of a suitable medium, particularly in the form of a disinfectant, have already been cited above.

In a third step, the medium acts on the connector area of the first connector 4, to be weakened in a partial area 20, and in particular also on the second material thereof. The preferred duration of the action depends on the geometry, the structure and the dimensions of the connector area 18 and can be, for example, between 5 seconds and 60 seconds. In this period of time, the medium acts on the connector area 18, e.g. by partially dissolving the second material, in such a way that the weakened structure of the connector area 18 forms in a partial area 20. The shape of the weakened structure is predetermined by the distribution of the second material in the first material and is therefore, for example, independent of a duration of action of the medium on the connector 4 also outside the partial area 20 to be weakened. The connector area 18 with the now generated weakened structure is still impermeable to fluid, as long as no external force acts on the connector area 18 in order to beak through the latter.

In a fourth step, the first connector 4 and the second connector 6 are connected to each other, in the present case via the respective second connection end 10 (FIG. 3). Here, the front male connector element 22 of the second connector 6 pushes (in the axial direction) through the weakened structure of the first connector 4. This leads to the connector area 18 of the first connector 4 being broken open into individual portions 24. The portions 24 are pressed (not shown) against the inner wall of the channel 12 of the first connector 4 by the front male connector element 22 and remain connected to the connector area 18 of the first connector 4 via the interruption(s) 26 in the weakened structure.

According to one variant, the first connector 4 and the second connector 6 are connected to each other by twisting. In this way, a torsional stress can be built up in the connector area 18, which leads to the weakened structure being torn open.

At the end of the fourth step, the first connector 4 is permeable to fluid, such that a fluid connection is permitted both to the associated fluid-conveying system and also to the second connector 6.

In an optional fifth step, the first connector 4 and the second connector 6 in the connected state are screwed onto each other by means of a union nut or are connected non-releasably to each other by means of a form-fit locking arrangement. If the first connector 4 and the second connector 6 are connected to each other by twisting, the two last steps can also be carried out simultaneously.

In a sixth step, the predetermined break point 28 of the second connector 6 is broken by a force exerted radially with respect to the flow direction S, optionally by means of a tool. This is done, for example, by bending the front male connector element 22. If the connector area 18 of the second connector 6 has no predetermined break point 28 but instead has a weakened structure generated by application of a medium, the weakened structure of the second connector 6 is also broken analogously. As a result, a fluid connection is produced between the first connector 4 and the second connector 6, and therefore also between the associated fluid-conveying systems.

To produce a connection between the first connector 4 and the second connector 6 of the connector system 2 according to the second embodiment of the invention, as shown in FIGS. 4 to 6, steps one to four described above are basically carried out.

In step four, however, when connecting the connectors 4, 6, it is not only the weakened structure of the first connector 4 that is broken open but advantageously also that of the second connector 6. When the first and second connectors 4, 6 are plugged together, in the present case in the axial direction or flow direction S, the connector area 18 of the second connector 6 is radially compressed, on account of the internal diameter of the front female connector element 16 of the first connector 4 being smaller by comparison with the external diameter of the front male connector element 22, with the result that a radial force acts on the connector area 18 and on the partial area 20 forming the weakened structure. This leads to the weakened structure being broken open by radial compression.

According to one variant, the weakened structure of the second connector 6 is broken open not by the compression in the front female connector element 16 of the first connector 4 but only when the front male connector element 22 of the second connector 6 reaches the channel 12 of the first connector 4. The channel 12 of the first connector 4 has a greater internal diameter than the front female connector element 16 of the first connector 4, such that the compressed connector area 18 of the second connector 6 relaxes again. This leads to the weakened structure being broken open by radial relaxation.

The undercut 30, formed by the front connector element 22 of the second connector 6, means that the fifth step described above is not needed here. Since the weakened structures 20 of the first connector 4 and of the second connector 6 are broken open in step four, the above sixth step is also not needed.

1. A connector for producing a fluid connection to a second connector, wherein the connector, in at least one partial area of a connector area, can form a weakened structure in order to be able to break open the connector in the connector area and in this way produce a fluid connection to the second connector,
wherein
the partial area of the connector area is designed in such a way that the weakened structure in said partial area can be generated by covering or irradiating the connector area with a medium.

2. The connector as claimed in claim 1, wherein the connector area has, in the partial area (20), a material which forms the weakened structure when the connector area as a whole is covered or irradiated with the medium.

3. The connector as claimed in claim 1, wherein the connector area has at least one first material and at least one second material, wherein the second material forms a partial area of the connector area and is less resistant to the medium than is the first material, such that the weakened structure is formed in the partial area of the connector area by contact of the second material with the medium.

4. The connector as claimed in claim 3, wherein the second material is a material chosen from a group of materials comprising polymethyl methacrylate, polycarbonate, polyethylene, polystyrene and polysulfone.

5. The connector according to claim 1, wherein the connector area is made from two different plastics, e.g. by the two-component technique.

6. The connector according to claim 1, wherein the connector area is designed as a closure area of the connector, which closes off the connector in a fluid-impermeable manner before it is broken open.

7. The connector according to claim 1, wherein the medium is a disinfectant.

8. The connector according to claim 1, wherein the medium for forming the weakened structure in the partial area of the connector area triggers a chemical reaction.

9. The connector according to claim 1, wherein, when the connector area is covered or irradiated with the medium, at least one stress crack is formed in said connector area in order to generate the weakened structure.

10. The connector according to claim 1, wherein, in relation to an intended flow direction (S) of a fluid through the connector, the partial area to be weakened extends, at least in part, radially and/or in the circumferential direction.

11. A connector system for producing a fluid connection between at least two fluid-conveying systems, said connector system having a first connector and a second connector, wherein
the first connector and the second connector can be connected to each other, and
at least the first connector, in a partial area of a connector area, can form a weakened structure in order to be able to break open the connector in the connector area and in this way produce the fluid connection to the second connector,
wherein
at least the first connector is designed as a connector as claimed in claim 1.

12. The connector system as claimed in claim 11, wherein, after it has been covered or irradiated with the medium, the connector area is designed to be broken open during connection of the first connector and the second connector.

13. The connector system as claimed in claim 12, wherein, after the connector area has been covered or irradiated with the medium, the connector area is able to be broken open by piercing the weakened structure during connection of the first connector and the second connector.

14. The connector system as claimed in claim 13, wherein the second connector, in order to pierce the weakened structure of the first connector, has a projection (22) which can be broken off from the second connector at a weakened area (28).

15. The connector system as claimed in claim 12, wherein after the connector area has been covered or irradiated with the medium, the connector area can be broken open by radial stressing of the weakened structure when the first connector and the second connector are connected and/or by a torsional stress when the first connector and the second connector are twisted relative to each other.

16. The connector system as claimed in claim 15, wherein one of the two connectors has a female connector element and the other has a male connector element which can be inserted into the female connector element in order to connect the two connectors, and in that an inner surface of the female connector element and an outer surface of the male connector element are designed in such a way that, by moving and/or twisting the two connector elements relative to each other, a force is generated that acts radially between the two connector elements and/or in the circumferential direction and causes the connector area to break open.

17. A method for producing a connection between at least two fluid-conveying systems by means of a first connector and a second connector, which each close off one of the systems, wherein the method comprises the following steps:
making available the first connector and the second connector, wherein in addition a weakened structure is to be provided in a partial area at least on the first connector, in at least one connector area, so as to be able to break open the first connector in this connector area and in this way produce a fluid connection to the second connector, and
bringing the first connector and the second connector together to produce the connection between the two connectors,
wherein
the weakened structure in the partial area is generated by covering or irradiating the connector area with a medium.

18. The method as claimed in claim 17, using a connector system.
